(19) United States
(12) Patent Application Publication
(10) Pub. No.: US 2014/0348962 A1
(43) Pub. Date: Nov. 27, 2014

(54) Method for Providing an Ingestible Nutritional Supplement to a Woman

(71) Applicants: Jonathan S. Thierman, Pikesville, MD (US); Ibrahim M. Hallaj, Waltham, MA (US)

(72) Inventors: Jonathan S. Thierman, Pikesville, MD (US); Ibrahim M. Hallaj, Waltham, MA (US)

(21) Appl. No.: 14/453,170

(22) Filed: Aug. 6, 2014

Related U.S. Application Data
(62) Division of application No. 13/206,675, filed on Aug. 10, 2011.

(51) Int. Cl. A23L 1/29; A61K 36/9068; A23L 1/308; A61K 9/00; A61K 9/20; A23L 1/304; A61K 36/185; A61K 31/717

(52) U.S. Cl. CPC A23L 1/293; A61K 36/185; A61K 36/9068; A61K 31/717; A61K 9/0095; A61K 9/20; A23L 1/304; A23L 1/308
USPC 424/756; 424/725; 514/57; 426/2

(57) ABSTRACT

An ingestible nutritional substance (supplement and the like) including vitamins, minerals and other ingredients beneficial to a woman and/or her child that a woman may take at a time of her life surrounding pregnancy. This includes by ingesting determined and suited types and amounts of ingredients, including at selected times, to best assist the woman and/or infant's needs at the respective times. The ingestible supplement can be formulated for drinking with appropriate nutritional and health benefits as well as with desired lifestyle and taste and aesthetic attributes. The dosing and packaging of the supplement to best suit a specific class of woman's and baby's needs are also described. Other aspects are directed to the preservation, storage and dispensation of one or more ingredients that may not be stable with one another during storage.

### RELATED APPLICATIONS

The present application is related to and claims the priority of U.S. Provisional Application No. 61/372,374, entitled “Ingestible Supplement for Women,” filed on Aug. 10, 2010, and is a divisional of and claims the priority of U.S. application Ser. No. 13/206,675 entitled “Ingestible Supplement for Women”, filed on Aug. 10, 2011, which are both hereby incorporated by reference.

### TECHNICAL FIELD

The present application relates to ingestible supplements and nutrients aimed to improve the health of women during and around the time of pregnancy.

### BACKGROUND

Certain nutrients, vitamins, minerals, and other nutritional materials are helpful to women who are pregnant. Prenatal vitamins are commonly found, which include some or many of these nutritional supplements. Each component of the prenatal vitamins purports to help the woman taking it with one or more aspects of her health and her baby's health. The prenatal vitamins are available as ingestible pills. The American Congress of Obstetricians and Gynecologists provides general guidelines on nutritional needs of pregnant mothers.

The references provided at the end of this disclosure give information that is known and helpful in establishing nutritional needs for women in the pregnancy cycle, and is incorporated herein by reference.

It should be noted that where an attempt is made to promote the health of a mother or baby in one or more aspects of their development cycles, this may rely on accepted views and studies available to the scientific and medical community. These views sometimes are refined or redirected, and an attempt is therefore also made to minimize or reduce risks from the lack of one or more nutritional materials in the diet of a mother and/or fetus.

Present ingestible supplements for mothers and expectant mothers are most commonly available as prenatal vitamins or pills (generally prenatal supplements). Current products do not typically differentiate between one woman or child and another. A woman is typically expected to swallow such prenatal vitamin pills once a day to provide her and her baby with important nutrients. The pills can be unsavory, bulky, and difficult to swallow. Also, pregnant mothers are sometime nauseated by the taste or feel of the prenatal pill. Other products have been available to fill the nutritional and health needs of a pregnant or gestating mother and child, but have not been provided in a scientifically-adapted form or have not been provided with specific methods for consumption that offer optimum health benefits to the mother and her baby. The present disclosure is directed to ingestible (generally drinkable) formulations and ways to make and consume the same that are more optimal for the health of pregnant women and their babies, or women who are expecting to become pregnant or who have recently been pregnant (e.g., breast feeding).

WARNING: The present disclosure is provided in the spirit of continuing development of useful products that may in the format provided herein or another format be healthful and useful for mothers and infants. However, it is important that members of the public not undertake self-treatment or consumption of new or experimental products without a thorough understanding of these products and of their own needs and limitations. Accordingly, any discussion herein is provided only for the purpose of developing the present application for new products and methods, and is not an instruction to consume anything whatsoever. Consumers should not make or consume things, including like those described herein, without having a proper discussion about the same with their physicians as the present application for patent is not intended as a prescription to make or ingest any combination of the things described herein. No product should be dispensed or consumed if it does not originate from a qualified and licensed vendor. Any and all medical advice must be sought from qualified health care providers only.

### SUMMARY

Some embodiments hereof are directed to formulations for nutritional supplements, prenatal vitamins, minerals, lifestyle ingredients and other ingredients of a product that may be consumed by a woman before, during, or after pregnancy, but most specifically targeting women around child-bearing times of their lives. Custom or specially-made formulations are provided in preferred examples below to correspond to a gestational phase of the woman's pregnancy (e.g., a trimester, or pre-pregnancy or breast feeding phases).

Accordingly, in some aspects, the present invention includes a nutritional sequence for gestating women, including those about to or planning to carry a child and are preparing their bodies for this process, the nutritional sequence including a plurality of ingestible nutritional formulations (for example one for each phase of the gestation), where each of said formulations includes a plurality of nutritional elements in respective dosages. The nutritional elements can include vitamins, minerals, herbs, aromatic compounds, natural and artificial ingredients beneficial to the health of the woman or her baby. The nutritional formulations being provided in formats and servings appropriate to the health of the woman and her developing baby. For example, suited to the woman and/or her baby's needs during the corresponding maternal or gestational phase of the mother and/or baby.

Embodiments hereof also provide optimal nutritional supplementation for women of differing ages, body weights, ethnicity, geographic domicile, or medical conditions.

Other aspects of the invention provide a method for assisting a mother and/or baby in or around the time of gestation, comprising determining a recommended dose suited for the woman to ingest, which can be a one-day recommended dose; dividing the one-day recommended dose into a plurality of portions thereof; wherein each of said plurality of portions comprises a pre-determined quantity of respective ingredients; and providing respective recommended time of day recommendations for the woman to ingest the corresponding plurality of portions.

Still other aspects hereof are directed to dividing the ingredients in the formulations of the supplements into appropriate units based on their nature. For example, if one or more ingredients are not stable for storage together the ingredients may be separated at the time of manufacture of the product into groups that are stored in the bottle of a drinkable product and only mixed or dispensed at or about the time of consumption. Mechanical separators and capsule units are described to achieve this end.

Yet further embodiments provide methods for making the above supplements, and even for dividing up a daily recommended amount of the supplement into a plurality of portions that are to be ingested at corresponding times of the day.

The present embodiments include drinkable forms that are provided in bottled or beverage containers, sometimes in groups that are convenient for women to take throughout their day or week or other period of time of their life around their gestation.

### IN THE DRAWINGS

FIG. 1 illustrates the time-dependence of a group of ingredients of an ingestible nutritional supplement, based on a gestational phase of a woman;

FIG. 2 illustrates in more detail an exemplary variation of the dosage of a given ingredient over the gestational phase of the woman;

FIG. 3 illustrates graphically the dosing of a user taking different quantities and at different times of day where a one-day recommended dose is split into a plurality of portions that can be taken separately for best results; and

FIG. 4 illustrates an exemplary system for providing multiple components of an ingestible supplement in drinkable form.

### DETAILED DESCRIPTION

The present disclosure provides examples of ingestible formulations for supplementing the diet and health of women and their babies. Some embodiments are directed to formulations for improving the health of pregnant women, including formulations that are adapted for pregnant women to take depending on the phase of their pregnancy. More specifically, provided herein are formulations for a plurality of ingestible nutritional supplements that are each suited for a corresponding plurality of phases of pregnancy, and even for phases that immediately precede and follow the pregnancy.

The present formulations may be taken in one or more format, depending on the user's choice and/or the advice of her medical care provider. For example, in a first embodiment, the supplements are taken as a drinkable beverage that is prepared in a bottling facility and sold in single- or multiple-serving containers (e.g., plastic drinking bottles, collapsible flexible liquid-carrying pouches, aluminum cans, and so on). In a second embodiment, the supplements are provided as a dissolvable solid or powder that can be mixed by the user with water or juice or milk or another beverage and ingested. In a third embodiment, the supplements are provided as a conventional swallowable pill form, which the user may swallow with water or another drink or food. In a fourth embodiment, the supplements are provided as chewable food items, snacks, pastes, spreads, or other edible forms.

In some aspects, the present formulations vary a dose or amount or concentration of a particular component thereof so that the component is delivered to the user in varying amounts at corresponding phases of the user's pregnancy or cycle. For example, in an embodiment providing folic acid, the amount of folic acid per serving can be made to vary with each trimester of her pregnancy. As mentioned above, if the user takes the supplements before and/or after pregnancy, the amounts of the components of the formulations can be made to suit these cycles of the woman's body and health as well.

The formulations herein can be marketed, packaged and advertised accordingly. While presentable as a sequence or series of supplements, the supplements can be labeled and sold together or separately. A supplement intended for women in their first trimester of pregnancy may be indicated as “T1” and those intended for women in the second trimester may be indicated as “T2” and so on (merely for convenience or for marketing purposes or other ease of reference).

It should be appreciated that some or all things ingested by a pregnant woman may be ingested in turn by her fetus. Therefore, this fact is considered in designing the supplements. For example, considerations to the development of the various body parts of the fetus are included in formulating some embodiments hereof so as to promote or assist or minimize the likelihood of harm in said development.

In some aspects, the supplements maintain the amount or concentration of a component thereof between an upper and a lower value. The upper and lower values may be dictated by guidelines used in the industry. So for example, an upper limit on the amount of vitamin A provided in each serving of the formulations may be adhered to avoid over-ingestion of this element by the user. Also, a lower limit on the amount of folic acid may be used in the making of the present formulations to provide at least a certain recommended baseline amount of folic acid to a user. The upper and lower limits used for a particular element or component may be constant throughout the pregnancy cycle, or may vary by phase of the cycle.

The following table (Table I) gives exemplary bases for making cycle-dependent formulations of the present supplements. The table shows various changes known or believed to occur in the mother and in the fetus, and some nutritional needs of the mother and/or fetus that can be addressed by a sequence of supplements given to the mother in the various cycles, including beginning just prior to pregnancy and continuing through each trimester of her pregnancy, and optionally extending into the post-partum and breast feeding cycle.

TABLE I

| | Pre- | First Trimester | Second Trimester | Third Trimester | Post-Partum/Breast |
|---|---|---|---|---|---|
| | Pregnancy | (T1) | (T2) | (T3) | Feeding |
| Changes in | Increased | Rising levels of | Rising Progesterone | Highest Pre- | Highest Calorie |
| Mother | Fertility | HCG cause | levels and increasing | eclampsia and | Requirement (500 extra |
| | Desired | nausea | girth cause | HTN risk | calories daily) |
| | | | constipation | | ACOG recommends at |
| | | | Mother's blood Volume Expands | | least 1000 mg Calcium |
| | | | and RBC production increases | | Daily |
| | | | Gestational DM highest risk | | |
| Nutrient | Herbal remedy | Ginger 1 g, lower | Fiber (25-30 grams | Calcium 1300 mg | Increased Fructose and |
| | (optional) | levels of iron | daily) | | carbs content |

| | supplement | Iron 30 mg, Vitamin C 85 mg | | Calcium 1000 mg |
|---|---|---|---|---|
| | | (helps with iron absorption) | | |
| | | Lower Fructose content | | |
| Fetal/Baby | Neural Tube Development | Bone, Hair, Fingernails | Most rapid neural | Continued brain |
| Development | (closes at 6 weeks) | development | and retinal | development |
| | Vitamin D may in high doses may | | development | Continued Growth |
| | cause birth defects in 1^{st} trimester | | Rapid Growth | |
| Nutrient | Folic Acid 800 mcg | Vitamin D 400 IU | Omega 3 650 mg, | Omega 3 650 mg, DHA |
| | Vitamin D 200IU | | DHA 250 mg | 250 mg |
| | | | Protein, | Increased Fructose and |
| | | | Increased | carbs content |
| | | | Calories from T2 | |
| | | | Vitamin D 400 IU | |

Baseline Prenatal Vitamin Provided
(Folic acid 400 mcg, Calcium 1000 mg, Iron 27 mg, DHA 250 mg, B6 1.9 mg, B12 2.6 mcg, etc)
Limit dose of Vitamin A and Vitamin D

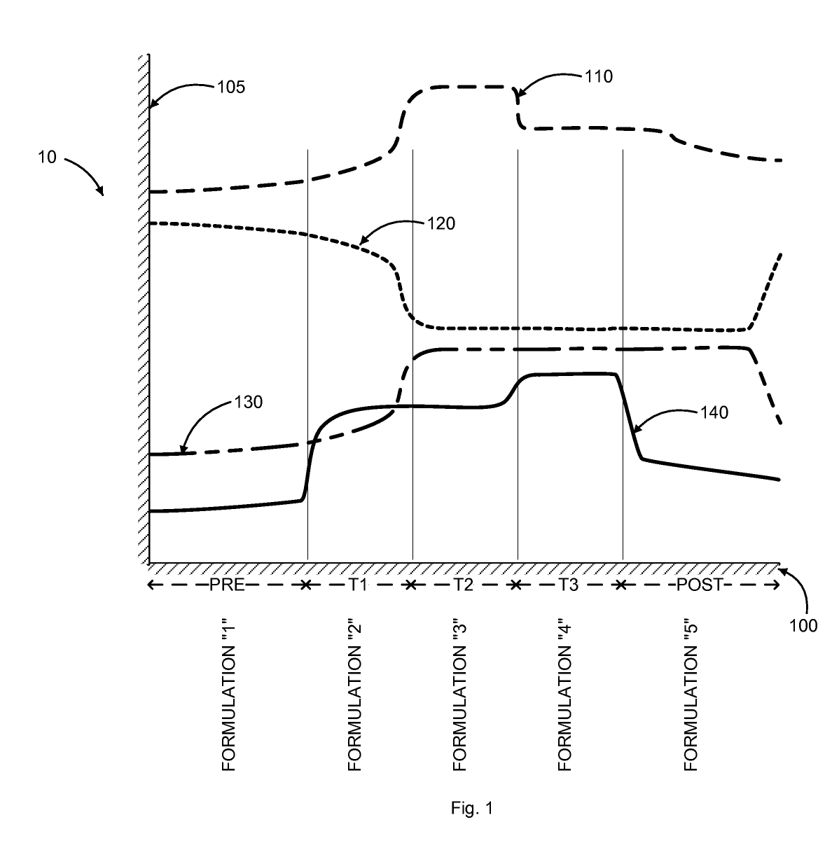

By way of illustration, reference is made to FIG. 1. The drawing shows a hypothetical and exemplary dose or concentration curve group 10, each curve corresponding to a particular nutrient, supplement element or vitamin or other thing useful for a mother to ingest during or around the time of her pregnancy. Time is shown along horizontal axis 100, and the amount, concentration or dose is shown on vertical axis 105. The figure is meant as conceptual in nature, and so actual doses and concentrations of components in a nutritional supplement may vary or be best illustrated on a logarithmic scale, or by other means.

A first curve 110 shows the recommended dosage or concentration of a first corresponding nutritional element that should be taken by a woman. It can be seen that the amount of this first nutritional element that is most appropriate for the woman varies in time. The horizontal time axis 100 is divided into phases of the maternal cycle, here indicated as PRE (pre-pregnancy), T1 (first trimester), T2 (second trimester), T3 (third trimester) and POST (post-partum) phases.

Similarly, the dose or concentration curve 120 shows the amount of a second nutritional element that is to be taken by the woman over the period of the maternal cycle, and other curves 130 and 140 for third and fourth nutritional elements are tracked as well.

From the above, it can be seen that a plurality of phases of the maternal cycle can be defined. For example, the phases of the maternal cycle may be divided along trimester divisions. These can of course be implemented in different time intervals, such as on a month-to-month, week-to-week, or other time measuring interval schemes without loss of generality. For each phase of the maternal cycle a corresponding respective formulation that includes an appropriate concentration, dose or amount of each nutritional element may be prepared and taken (ingested) by the woman during that phase of her cycle. The curves 110, 120, 130, 140 may thus be approximated with step functions that simplify and approximate the dosage or concentration or amount of each nutritional element that is to be included in the nutritional supplement formulation designed to be taken during the corresponding phase of the woman's maternal cycle. In the example of FIG. 1, five distinct formulations are to be prepared (Formulation 1 . . . Formulation 5). The formulations can be packaged and identified so that a user can determine which one she is supposed to take during each of the respective corresponding phases of her maternal cycle.

The taste, appearance, smell and other attributes of the present ingestible supplement are preferably pleasing to a woman of child bearing age and disposition. For example, the taste of the supplement may be selected to contain flavors and textures to suit the user. In some embodiment, familiar flavors are employed or added to the supplement for drinking as a beverage. For example, fruity flavors (e.g., citrus varieties, berry varieties, apple, pear, melon, kiwi, mango, etc.) or combinations thereof are included, whether by natural ingredients or by artificial flavoring to achieve the desired effect. Odor and mouth feel are also suitably selected so that a beverage based product is pleasant to drink. The product may have an overall sweet, sour, tangy, or other familiar or interesting taste, or an apparent combination of tastes. The color may also be made to suit the overall impression delivered by the product, for example by providing substantially clear liquid appearance in some embodiments. In some embodiments the color of the liquid product generally resembles the color of a fruit associated with the ingredients or flavor of the product. For example, a lime-flavored product may include citrus ingredients (natural and/or artificial) and may be substantially yellow, green, or orange in color. A clear, thin, sports-drink type product or beverage may have a relatively low viscosity (less than about 15 or 20 centipoise at room temperature).

In yet other embodiments, an umami flavor base is employed. This type of product still delivers the present nutritional and health advantages, but has a savory taste. Milk-based, soy-based, chocolate-based, caramel-based, creamy, or coffee-based flavor varieties and comprehended. These may be optically opaque or turbid in appearance to complement the taste of the product. Also, this type of liquid may have a greater viscosity than that given above.

Now a preferred but exemplary embodiment is provided describing ingredients that are employed in the embodiment. As stated before, these ingredients may be provided in quantities appropriate for a particular stage of pregnancy or of a woman's gestational cycle, including before and after pregnancy.

One or more embodiments provide a formulation including: Calcium Lactate, di-Alpha; Tocopherol Acetate (Vitamin E); Ascorbic Acid (Vitamin C); Ferrous Gluconate; Ginger Root; Zinc Aspartate; Biotin; Pyridoxine; Hydrochloride (Vitamin B-6); Niacinamide (Vitamin B-3); Vitamin A Palmitate; Cholecalciferol (Vitamin D-3); Thiamin; Hydrochloride (Vitamin B-1); Folic Acid; Cyanocobalamin (Vitamin B-12); Potassium Iodide.

In other embodiments, the present ingestible supplements may include carbohydrates, sugars, vitamins, and other useful minerals and substances. In a specific example, only provided for illustration where quantities are given, this includes: Vitamin A (4000 IU); Vitamin C (100 mg); Vitamin D-3 (400 IU); Vitamin E (30 IU); Vitamin B-1 (1.7 mg), Thiamin; Vitamin B-2 (1.7 mg), Riboflavin; Vitamin B-3 (20 mg), Niacin; Vitamin B-6 (20 mg), Pyridoxine; Folate (800 mcg); Vitamin B-12 (8 mcg), Cyanobobalamin; Calcium (200 mg); Iron (18 mg); Zinc (15 mg); Biotin (300 mcg); Iodine (150 mcg); as well as selected botanicals such as Ginger (125 mg) and/or docosahexaenoic acid (DHA) such as micellized water soluble DHA (200 mg).

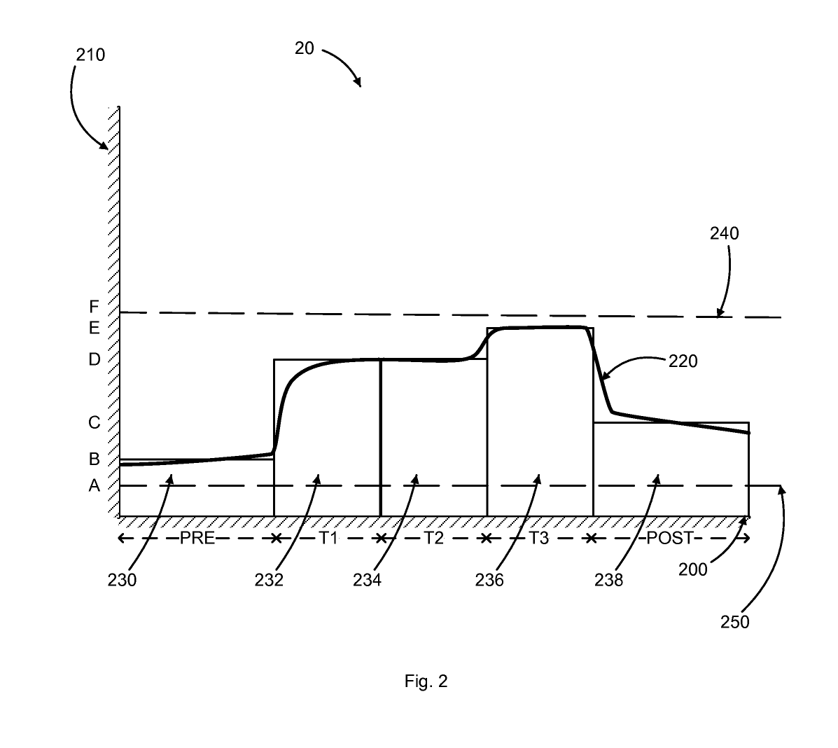

FIG. 2 looks at the concentration of a single nutritional element 20 from among the several nutritional elements that are included in a nutritional supplement. The time axis 200 is horizontal, and the amount, concentration, or dose axis 210 is vertical. The optimum recommended amount of this nutrient for a woman during the phases of her maternal cycle is shown by curve 220, which is for example the same as curve 140 from the previous figure. Here, a discrete number of phases of the maternal cycle are defined, and for each a determined dose or concentration or amount of the nutrient is defined.

Specifically, for the pre-pregnancy phase, an amount B (which can be milligrams per serving) is included in the formulation to be taken by the woman during the PRE phase. A greater dose D is defined for the second (T1) phase. The third phase (T2) was determined to require about the same dose D. The fourth phase (T3) was determined to require a yet greater amount E of the nutrient per serving. And finally, in the POST phase it was determined that a reduced dose C was best. In this way, several formulations, each containing the determined best amount of the nutrient are prepared and offered to the woman as a sequence or series of nutritional supplements for her to ingest, one formulation for each phase of her pregnancy and maternal cycle. The doses are shown as bar chart or histograms 230, 232, 234, 236 and 238 corresponding to the phases PRE, T1, T2, T3 and POST. Each of the other nutritional supplement components can be similarly identified and amounts for each of these components defined for taking during each phase of the maternal cycle. In the end, a multi-phase series of supplements are given to a woman so that she can take one type of formulation for the duration of one of the phases of her pregnancy, then she switches to the next formulation, and so on.

It should be appreciated that a minimum dose can be defined as shown by the dashed horizontal line 250, e.g. having an amount A; and also, a maximum dose as shown by line 240 can be defined. The minimum and maximum amounts recommended can be derived from recommendations of a medical or nutritional organization, government regulation, or other empirical data. In this way, the dosage or amount of each nutritional supplement component can vary as recommended for a woman in a given phase of her cycle without departing from the dosage guidelines.

Some or all embodiments hereof can include a liquid multivitamin formula. A formulation for the ingredients of the formulation can include any or all of: vitamins, minerals, botanical extracts, botanical infusions, fiber, probiotics, and other natural or artificial ingredients. Other ingredients may be added to enhance the taste (e.g., sugars) or appearance (e.g., coloring).

As discussed above, some present embodiments are provided in a drinkable (liquid) form that is consumed by a woman in the same way she would consume a juice drink or health beverage or nutritious milkshake, etc.

Variations of the above examples are comprehended as well. For example, different age groups of women may be provided with special formulations appropriate for their age groups. This may be especially useful for women near menopause or women that are especially young. Also, the formulations and recommended dosage may be varied depending on a woman's body weight, for example providing more of the product to women whose body weight is greater. Additionally, the amounts provided to a woman may depend on her medical history, specific allergies or nutritional needs, or other particular attributes of the woman.

In other embodiments, the ingestible supplements may be customized to the climate or environment of the women consuming them. For example, women from certain cultures, climates, or regions of the world may naturally lack certain ingredients, whether because of the scarcity of the ingredients in their natural local diets or because of other cultural or economic factors. Therefore, the present products may be tailored for providing ingredients needed by such women in corresponding amounts.

Alternatively, it may be determined that seasonal changes (in temperature, humidity, and so on) advise women to take certain ingredients in certain amounts. Currently, all women's multi-vitamins and prenatal supplements are generically produced and do not take into account the above factors such as the race, weight, geographic domicile, or seasonal needs of the women consuming them. And as stated earlier, current products are generally provided in lumped (pill or single serving) form that is input into the woman's body substantially all at once.

As mentioned before, a drinkable (substantially liquid) ingestible supplement may be provided to users in a beverage form. The beverage may be provided to users in a packaged beverage container, e.g., a plastic bottle, glass bottle, aluminum can, or fibrous or waxed paper container, disposable cups or cartons, etc. Where a user purchases a number of beverage containers to cover a supply for several days or an extended time, the beverage containers may be included in a bundled packaging format. A plurality of individual beverage containers may be bundled into groups corresponding to a nutritional or biological quantity or other convenient or logical amount of the beverage and/or number of individual containers. For example, a week-long supply may be provided in multiples of seven (7) so that one beverage container of the ingestible supplement may be consumed per day. Cases of thirty (30) daily servings are also contemplated so as to last the woman a whole month.

In some embodiments, the serving size is about 12 to 24 fluid ounces. In a particular embodiment, the serving size is about 16 fluid ounces. A recommended daily dose of the above may be contained in a single beverage container. In other embodiments, a recommended daily dose is contained in a plurality of beverage containers. For example, in two containers, one for consumption at an early time of day, and another for consumption at a later time of day.

More than two separate sub-doses of the daily recommended amount may be packages separately to be taken throughout a day as well. So, for example, in one embodiment, the overall daily amount of ingestible supplements may be divided into three separate servings, which total to the desired daily dose. Each of the servings may be taken at or around the time of a daily main meal (breakfast, lunch, dinner) or between meals. This way, the ingestion of each of the three portions (and more or less than three are possible as well) can be designed to optimize the introduction of the ingredients into the woman's body. More specifically, the ingredients may be allocated to each of the portions so that they are taken at a time of day that is more appropriate or optimal for each ingredient. For example, if a person wakes up in the morning lacking hydration, then the morning dose may include more hydrating ingredients than the other portions. If a person can benefit in the middle of her day from energy-providing ingredients to get her through the day, then carbohydrates or sugars may be more highly concentrated at the mid-day portion. If Iron or other ingredients are best taken before bed (as opposed to when the woman awakes) then it can be best to reduce the amount of Iron or other ingredient in the morning and mid-day portions and allocate those ingredients towards the end of day portion.

It should be appreciated that the sizes of the portions taken throughout the day may vary and do not need to be the same. For example, the mid-day portion may be larger than the others, or the beginning of the day portion may be larger than the others, etc.

The time separation between drinking the first and second and third portions of the recommended daily dose may be prescribed as part of the instructions for the user who purchases or uses the product. Again, it is noted that the present product or product suite or method for supplying the nutritional supplement and prenatal vitamins may include one, two, three or another number of sub-doses or portions to be taken during a day. The time separation between the portions may not necessarily need to be uniform or the same for each woman. Some women are more susceptible to nausea early in the morning, and they may determine for themselves the timing of taking the substance. A physician or pharmacist may also advise on the optimal way to take the doses for a given patient.

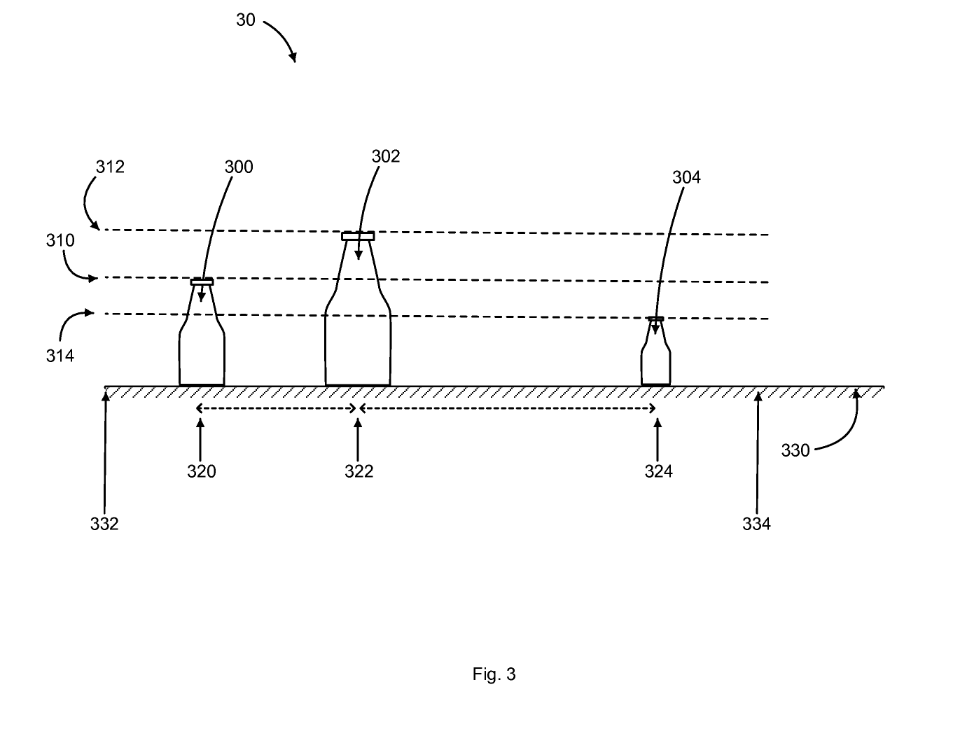

FIG. 3 illustrates in a graphical way a method 30 for providing the present ingestible supplements to women who are pregnant or near/after their pregnancies. The horizontal axis 330 represents a time line, which may extend from an early time of day 332 when the woman awakes and extending through a late time of day (night) 334 when the woman goes to sleep. In this example, three portions of the ingestible, e.g. drinkable, supplement are provided 300, 302, 304.

The first portion 300 contains a first amount (quantity) 310, which may include a first combination of ingredients as described above. The second portion 302 contains a second amount (quantity) 312, which may include a second combination of ingredients. The third portion 304 contains a third amount (quantity) 314, which may include a third combination of ingredients.

Each of the three portions 300, 302, 304 is taken at respective times of the day. The three portions 300, 302, 304 are ingested throughout the day at a schedule of the woman's choosing or as recommended by the manufacturer or a health practitioner. The first (perhaps morning) dose 300 is taken at a first time 320. The second (perhaps mid-day) dose 302 is taken at a second time 322. The third (perhaps evening) dose 304 is taken at a third time 334. In this way, the optimum amount and type of ingredients may be supplied to the user at the appropriate times of day.

In another aspect, there may be presented in the present ingestible supplements small swallowable capsules containing useful substances that can be digested or taken up by a woman's body over an extended time period. For example, if a sudden uptake of a vitamin or mineral (e.g., iron) is upsetting to a woman's stomach, she may choose a variety of the present products that are “gentle” on the digestive system and do not cause as much nausea or constipation as iron can sometimes do. Here, the ingredients where iron is present can be encapsulated in shells, capsules, pellets, or coatings that dissolve gradually over time and permit the ingredient, for example iron, to be taken slowly over time instead of all at once. The size of the small pellets may be designed, and the product may include several different types of small pellets in suspension in the drinkable form supplements, which each contains certain ingredients with engineered release dynamics when ingested.

For certain formulations, one or more ingredients may be unstable when exposed to oxygen, water, or other chemical substances found in the formulation. In this case, embodiments of the present invention can include ways to avoid or reduce the mixing or comingling of incompatible ingredients with one another.

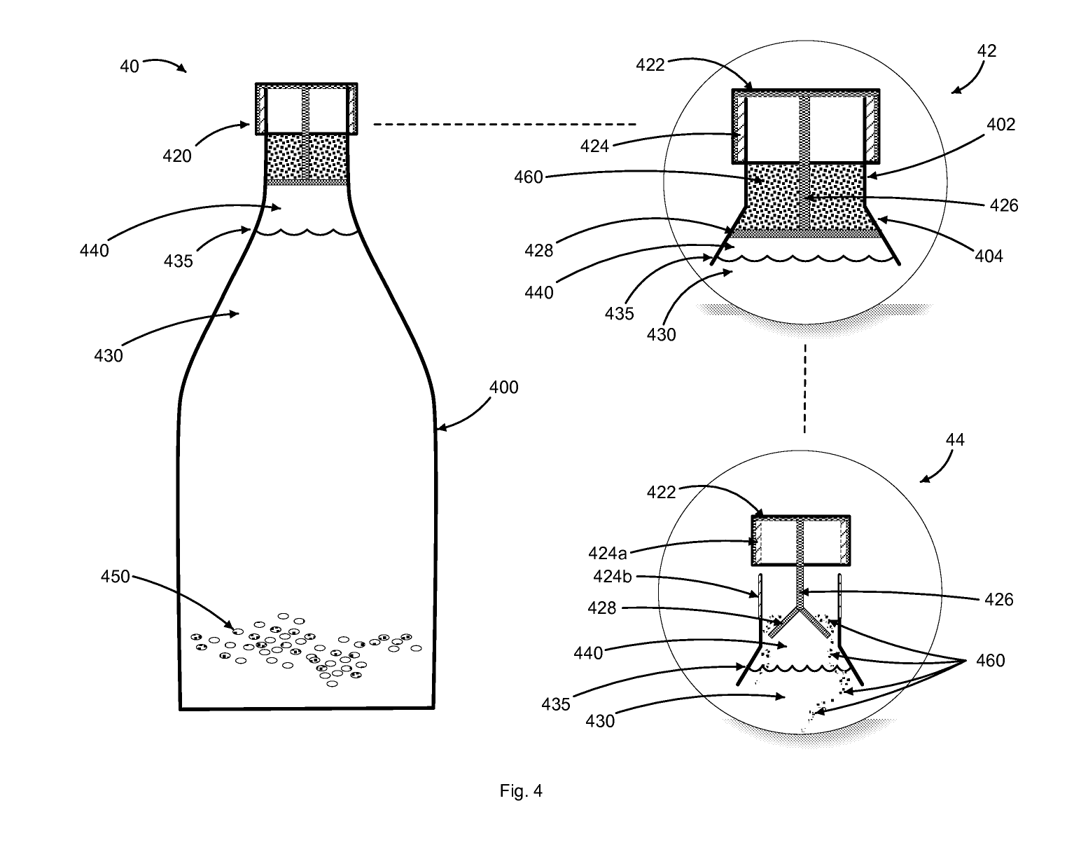

FIG. 4 illustrates an exemplary mechanism for storing and providing to a user a drinkable ingestible supplement such as those described above when one or more ingredients are to be held separate from one another. The reason for separately holding the one or more ingredients may be chemical or nutritional stability as stated before. It is noted that all active ingredients of the supplement may be stored in a compartment separate from the drinkable fluid, or only some or only one may be so kept separate in said compartment. It is also noted that more than one separation compartment or chamber may be employed if a plurality of distinct mechanically separate compartments are desired or required. Similar principles to those discussed here for the sake of explanation may be devised and employed by those skilled in the art.

A bottle 40 (generally, a container suitable for holding and dispensing a liquid-based nutritional supplement) is provided, and may be provided as part of a multi-bottle set or pack of bottles 40 to be consumed by the end user. The bottle 40 includes a housing such as a plastic or glass or aluminum shell 400 that holds the liquid contents 430 thereof. A cap section 420 seals the contents of bottle 40 by mating to the neck or opening of housing 400. Threaded (screw top) or friction (cork) means may be used to secure the cap section 420 to the bottle housing or shell 400. A void space or gas volume 440 generally exists at the upper volume of bottle 40 because the density of the gas 440 left in the bottle at the time of bottling is less than that of the liquid 430. When held upright as shown, a meniscus 435 generally delineates the interface between the gas volume 440 and the liquid 430 below it.

In some embodiments, one or more liquid or solid ingredients of the ingestible supplement may be manufactured into small ingestible pills, pellets, beads, or capsules 450. The ingestible capsules 450 may be in suspension in the liquid 430 and may float, sink, or be neutrally buoyant in the fluid 430. The beverage as a whole may be shaken at the time of consumption so as to mix the ingestible capsules 450 in the drinkable product where they are swallowed and processed in the user's digestive system because the shells or coatings of ingestible capsules 450 are soluble or otherwise destroyed not in the drink product but in the user's body so that they can be consumed.

More than one type of ingestible capsule 450 may be provided within a same drinkable product. For example, if several species of ingredient of the drinkable product are unstable for the duration of the product's shelf life, a first such ingredient may be encapsulated in a first species of ingestible capsule, and a second such ingredient may be encapsulated in a second species of ingestible capsule, and so on. Also, more than one ingredient may be packed into a single species of ingestible capsule if such more than one ingredients are compatible with or stable with one another.

A closer view of the cap section 420, depicting a closed bottle, is shown in view 42. An outer cap 422 includes securing means such as threads 424 or friction elements or break tabs so securely hold on to and seal the contents of the bottle. The cap 422 is secured to an upper (neck) portion 402 of the bottle, and the bottle generally widens below at section 404. A diaphragm 428 separates ingredients 460 above the diaphragm from the rest of the contents of the bottle below, and the diaphragm is located at a depth in the bottle so that the diameter of the diaphragm is somewhat greater than the narrow diameter of the neck of the bottle. A seam, joint, or plurality of molded bumps or protrusions on the inner surface of the bottle near its neck may hold the diaphragm 428 in place during storage and may facilitate actuation of the diaphragm according to the steps below.

Of present interest in this aspect is the exemplary apparatus for separating some or all ingredients 460 of the ingestible supplement prior to consumption. A plunger stalk 426 is mechanically coupled to and in solid communication with the outer cap 422. Therefore, movement of the cap 422 by a user would translate into a corresponding movement of the plunger stalk 426, including when the cap 422 is twisted and including when the cap 422 is pulled off to open the bottle. A semi-rigid (e.g., plastic, rubber, cardboard, wax, or fibrous composite) piston or diaphragm 428 is coupled to the plunger stalk 426 at or near a central portion of diaphragm 428. Hence, when a user opens the bottle for consumption, she twists off the threaded cap 422, which pulls the cap 422 and connected plunger stalk 426 upwards, which pulls the central portion of diaphragm 428 upwards as well.

View 44 shows a depiction of a bottle cap as described above being opened. As explained, threaded cap 422 is unscrewed and lifted off of the threaded neck of the bottle. Mating threads 424a and 424b are provided on the inside surface of the cap 422 and outside surface of the bottle's neck, respectively. Plunger stalk 426 is pulled up and out of the bottle with the cap 422, and causes diaphragm 428 to buckle, bend, distort, tear, or otherwise deform as it is pulled out of the neck of the bottle, which is generally narrower than the area of the diaphragm 428. When the diaphragm so distorts, it no longer supports the ingredients previously held in isolation above the diaphragm. The contents 460 drop into the liquid 430 in the bottle under the force of gravity.

The ingredients 460 may be in powder form, solid form, liquid form, or another form as appropriate. The ingredients become dissolved in or mixed with or in suspension in the liquid 430 when the cap of the bottle is removed by the user. The user then drinks the contents of the bottle, including the liquid contents 430 and the ingredients 460 that were previously stored in the separating cap section 420. In this way, even if a complete supplement includes one or more ingredients that are not stable in combination for the duration of the product's shelf life, they can be combined just prior to consumption.

Numerous other ways of combining separate ingredients in a drinkable supplement product would be appreciated by those skilled in the art, some examples of which are described in related work by the present applicants, such work and concepts being hereby incorporated by reference. Some examples where a pre-natal ingestible supplement may employ separation or isolation means to separate one or more ingredients includes in the provision of DHA to a woman in a product containing minerals such as iron, zinc or other metals. The DHA may be stored in a separate chamber, in liquid or in solid or powdered form and only added to the bulk fluid 430 containing iron at the time of consumption. Conversely, DHA may be present in the bulk fluid 430 and the iron or other metals may be kept in isolation above the diaphragm 428.

As discussed above, The present invention should not be considered limited to the particular embodiments described above, but rather should be understood to cover all aspects of the invention as fairly set out in the attached claims. Various modifications, equivalent processes, as well as numerous structures to which the present invention may be applicable, will be readily apparent to those skilled in the art to which the present invention is directed upon review of the present disclosure. The claims are intended to cover such modifications and equivalents.

What is claimed is:

1. A method for providing an ingestible nutritional supplement to a woman, comprising:
determining a one-day recommended dose suited for the woman;
dividing said one-day recommended dose into a plurality of portions of said one-day recommended dose;
wherein each of said plurality of portions comprises a pre-determined quantity of respective ingredients; and
providing a respective recommended time of day for ingestion of each of said plurality of said portions, said respective times of day not being at a same time as one another.

2. The method of claim 1, determining said one-day recommended dose comprising a determination based on said woman's body mass.

3. The method of claim 1, determining said one-day recommended dose comprising a determination based on said woman's geographic domicile.

4. The method of claim 1, determining said one-day recommended dose comprising a determination based on a seasonal time of year in which the supplement is being taken.

5. The method of claim 1, further comprising packaging said plurality of portions in individual packages that can be consumed by said woman at selected times during the day.
